(19) United States
(12) Patent Application Publication
(10) Pub. No.: US 2015/0322120 A1
(43) Pub. Date: Nov. 12, 2015

(54) SODIUM CHANNEL SENSITIVE CONOPEPTIDES AND ANALOGS, INCLUDING COMPOSITIONS AND METHODS THEREOF

(71) Applicants: University of Utah Research Foundation, Salt Lake City, UT (US); Janssen Pharmaceutical NV, Breese (BE)

(72) Inventors: Julita Imperial, Midvale, UT (US); Brad Reed Green, Sandy, UT (US); Joanna Gajewiak, Salt Lake City, UT (US); Minmin Zhang, Sandy, UT (US); Doju Yoshikami, Salt Lake City, UT (US); Grzegorz Bulaj, Salt Lake City, UT (US); Baldomero Olivera, Salt Lake City, UT (US); Alan Wickenden, San Diego, CA (US); Yi Liu, San Diego, CA (US)

(73) Assignees: University of Utah Research Foundation, Salt Lakee City, UT (US); Janssen Pharmaceutica NV, Breese (BE)

(21) Appl. No.: 14/394,719

(22) PCT Filed: Apr. 17, 2013
(86) PCT No.: PCT/US13/37030

§ 371 Date: Oct. 15, 2014

Related U.S. Application Data
(60) Provisional application No. 61/625,318, filed on Apr. 17, 2012.

(51) Int. Cl. C07K 14/435

(52) U.S. Cl. CPC C07K 14/43504

(57) ABSTRACT

The present invention relates to conopeptides that are naturally available in minute amounts in the venom of the cone snails or analogous to the naturally available peptides, and which block the sodium channels.

### FIELD OF THE INVENTION

The present invention relates to conopeptides and analogs thereof that can control or otherwise affect behavior of voltage-gated sodium channels, such as Nav 1.1-1.7 channels. Many conopeptides are found in minute amounts in the venom of cone snails (genus Conus). As such, the present invention involves the fields of chemistry, biochemistry, molecular biology, and medicine among others.

### BACKGROUND OF THE INVENTION

All publications, patents, and other materials used herein are incorporated by reference.

The venom of marine gastropods in the genus Conus has yielded numerous structurally and functionally diverse peptidic components. The increasing variety of bioactive peptides identified in cone snail venoms has provided insight into the seemingly endless variety of directions taken by Conus species in evolving neuroactive molecules to suit their diverse biological purposes.

The bioactive peptides in Conus (“conopeptides”) are classified into two broad groups: the non-disulfide-rich and the disulfide-rich. The latter are conventionally called conotoxins. The non-disulfide-rich class includes conopeptides with no cysteines (contulakins and conorfamides), and conopeptides with two cysteines forming a single disulfide bond (conopressins and contryphans). The conopeptides that comprise the disulfide-rich class have two or more disulfide bonds. Among the major classes of molecular targets identified for these structurally diverse conopeptides are members of the voltage-gated and ligand-gated ion channel superfamilies.

The structure and function of a number of these peptides have been determined. Three classes of targets have been elucidated: voltage-gated ion channels; ligand-gated ion channels, and G-protein-linked receptors.

Conus peptides which target voltage-gated ion channels include those that delay the inactivation of sodium channels, as well as blockers specific for sodium channels, calcium channels and potassium channels. Peptides that target ligand-gated ion channels include antagonists of NMDA and serotonin receptors, as well as competitive and noncompetitive nicotinic receptor antagonists. Peptides which act on G-protein receptors include neurotensin and vasopressin receptor agonists. The pharmaceutical selectivity of conotoxins is at least in part defined by specific disulfide bond frameworks combined with hypervariable amino acids within disulfide loops.

Voltage-gated sodium channels are found in all excitable cells including myocytes of muscle and neurons of the central and peripheral nervous system. In neuronal cells, sodium channels are primarily responsible for generating the rapid upstroke of the action potential. In this manner sodium channels are essential to the initiation and propagation of electrical signals in the nervous system. Proper and appropriate function of sodium channels is therefore necessary for normal function of the neuron. Consequently, aberrant sodium channel function is thought to underlie a variety of medical disorders including epilepsy, arrhythmia, myotonia, and pain.

There are currently at least nine known members of the family of voltage-gated sodium channel (VGSC) alpha subunits. Names for this family include SCNx, SCNAx, and Navx.x. The VGSC family has been phylogenetically divided into two subfamilies Nav1.x (all but SCN6A) and Nav2.x (SCN6A). The Nav1.x subfamily can be functionally subdivided into two groups, those which are sensitive to blocking by tetrodotoxin (TTX-sensitive or TTX-s) and those which are resistant to blocking by tetrodotoxin (TTX-resistant or TTX-r).

The Nav1.7, alternatively written as NaV1.7, (PN1, SCN9A) VGSC is sensitive to blocking by tetrodotoxin and is preferentially expressed in peripheral sympathetic and sensory neurons. The SCN9A gene has been cloned from a number of species, including human, rat, and rabbit and shows about 90% amino acid identity between the human and rat genes.

### BRIEF DESCRIPTION OF THE DRAWINGS

FIGS. 1A and 1B show concentration response curves for C. geo1 analogs against hNaV1.7. FIG. 1A: IC₅₀ value for the internally-truncated synthetic peptide C. geo1[des-Ser34] was calculated as 1.8 μM. FIG. 1B: Concentration-response curves were repeated on the full-length peptide, in addition to the analog containing the amino-butyric acid isosteric replacement at position 24 (C. geo1[C24Abu]).

### DETAILED DESCRIPTION

In describing and claiming the present invention, the following terminology will be used in accordance with the definitions set forth below.

The singular forms “a,” “an,” and, “the” can include plural referents unless the context clearly dictates otherwise. Thus, for example, reference to “a peptide” can include reference to one or more of such peptides, and reference to “the analog” can include reference to one or more of such analogs.

As used herein, “subject” refers to a mammal that may benefit from the administration of a composition or method according to aspects of the present disclosure. Examples of subjects include humans, and may also include other animals such as horses, pigs, cattle, dogs, cats, rabbits, and aquatic mammals.

As used herein, the term “peptide” may be used to refer to a natural or synthetic molecule comprising two or more amino acids linked by the carboxyl group of one amino acid to the alpha amino group of another. A peptide of the present invention is not limited by length, and thus “peptide” can include polypeptides and proteins. Amino acid sequences are written left to right in amino to carboxy orientation, respectively.

As used herein, the term “isolated,” with respect to peptides, refers to material that has been removed from its original environment, if the material is naturally occurring. For example, a naturally-occurring peptide present in a living animal is not isolated, but the same peptide, which is separated from some or all of the coexisting materials in the natural system, is isolated. Such isolated peptide could be part of a composition and still be isolated in that the composition is not part of its natural environment. An “isolated” peptide also includes material that is synthesized or produced by recombinant DNA technology or that is synthetically created.

Unless defined otherwise, all technical and scientific terms used herein have the same meaning as is commonly understood by one of skill in the art to which the invention belongs.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint without affecting the desired result.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### DETAILED DESCRIPTION

The present disclosure provides novel peptides showing activity in blocking sodium channels, including various associated compositions and methods. More particularly, these peptides block at least voltage-gated sodium channels. Much of the description herein pertains to NaV1.7 sodium channels; however it is understood that the present scope includes any sodium channels, voltage-gated or otherwise, that are affected by the present peptides. It is noted that these peptides are derived from the venom of Conus geographus snails using a combination of venom fractionation, sequencing, cloning and transcriptomics, and that the present scope additionally includes the naturally occurring peptides, completely or partially synthesized peptides, and related analogues thereof.

The present peptides can be identified by isolation from Conus venom. Additionally, the present peptides can be identified using recombinant DNA techniques by screening cDNA libraries of various Conus species using conventional techniques such as the use of reverse-transcriptase polymerase chain reaction (RT-PCR) or the use of degenerate probes. Primers for RT-PCR are based on conserved sequences in the signal sequence and 3′ untranslated region of the propeller peptide genes. Clones that hybridize to these probes can be analyzed to identify those which meet minimal size requirements, i.e., clones having approximately 300 nucleotides (for a precursor peptide), as determined using PCR primers that flank the cDNA cloning sites for the specific cDNA library being examined. These minimal-sized clones can then be sequenced. The sequences are then examined for the presence of a peptide having the characteristics noted above for peptides. The biological activity of the peptides identified by this method is tested as described herein, in U.S. Pat. No. 5,635,347, or conventionally in the art.

The present peptides are sufficiently small to be chemically synthesized by techniques well known in the art. The peptides are synthesized by a suitable method, such as by exclusively solid-phase techniques (Merrifield solid-phase synthesis), by partial solid-phase techniques, by fragment condensation or by classical solution couplings. Suitable techniques are exemplified by the disclosures of U.S. Pat. Nos. 4,105,603; 3,972,859; 3,842,067; 3,862,925; 4,447,356; 5,514,774; 5,591,821 and 7,115,708, each incorporated herein by reference. In one non-limiting aspect, a solid peptide synthesis protocol can be optimized using a low preloaded Wang resin in combination with pseudoproline Fmoc-Tyr(tBu)-Thr(ψ^{Me,Me} pro)-OH to obtain enhanced purity for the crude linear products.

Various of the peptides described herein can also be obtained by isolation and purification from specific Conus species using the techniques described in U.S. Pat. Nos. 4,447,356; 5,514,774 and 5,591,821, the disclosures of which are incorporated herein by reference. The peptides described herein can also be produced by recombinant DNA techniques well known in the art.

Peptides produced by chemical synthesis or recombinant DNA techniques can be isolated, reduced if necessary, and oxidized to form disulfide bonds. One method of forming disulfide bonds is the air oxidation of the linear peptides for prolonged periods under cold room temperatures or at room temperature. This procedure results in the creation of a substantial amount of the bioactive, disulfide-linked peptides. The oxidized peptides can be fractionated using reverse-phase high performance liquid chromatography (HPLC) or the like, to separate peptides having different linked configurations. Thereafter, either by comparing these fractions with the elution of the native material or by using an assay, the particular fraction having the correct linkage for maximum biological potency can be determined. However, because of the dilution resulting from the presence of other fractions of less biopotency, a somewhat higher dosage may be beneficial.

Muteins, analogs, or active fragments of the peptides described herein are also contemplated. Derivative muteins, analogs or active fragments of the present peptides can be synthesized according to known techniques, including conservative amino acid substitutions, such as outlined in U.S. Pat. No. 5,545,723 (see particularly col. 2, line 50 to col. 3, line 8); U.S. Pat. No. 5,534,615 (see particularly col. 19, line 45 to col. 22, line 33); and U.S. Pat. No. 5,364,769 (see particularly col. 4, line 55 to col. 7, line 26), each incorporated herein by reference.

In one aspect of this invention, a novel peptide having 7 cysteine residues is provided, where the peptide has a sequence of X₁X₂C X₄X₅X₆X₇X₈X₉C X₁₁X₁₂X₁₃X₁₄X₁₅X₁₆CCX₁₉X₂₀X₂₁C X₂₃C₂₄X₂₅X₂₆X₂₇X₂₈Ĉ (SEQ ID 033). It is noted that X₁₋₂, X₄₋₉, X₁₁₋₁₆, X₁₉₋₂₁, X₂₃, and X₂₅₋₂₈ can each independently be any amino acid that allows functionality of the resulting peptide, and that the spacing of the cysteine residues is preserved. C₂₄ is cysteine or a substituted cysteine, and ̂ is a carboxylated C-terminus, as is discussed further herein.

In one aspect, X₂₇ can be lysine or glycine. In another aspect, X₆ can be hydroxyproline or alanine. In yet another aspect, X₂₃ can be aspartic acid, gamma-carboxyglutamic acid, or asparagine. In a further aspect, X₂₅ can be tyrosine or aspartic acid.

In one aspect, this invention provides peptides having a sequence GWCGDOGATC GKLRLYCCSG FCX₂₃C₂₄X₂₅TKTC-X₃₀̂ (SEQ ID 001), where O is hydroxyproline, X₂₃ is aspartic acid, asparagine, or carboxyglutamic acid, C₂₄ is cysteine or a substituted cysteine, X₂₅ is tyrosine or aspartic acid, X₃₀ is a peptide from 0 to 6 amino acids, and ̂ is a carboxylated C-terminus. In one aspect, the peptide can be an isolated peptide. In another aspect, the peptide can be a synthetic peptide. Numerous synthesis protocols and techniques are known, and any such technique that can be utilized to generate synthetic peptides is considered to be within the present scope. For example, in one aspect solid peptide synthesis can be utilized.

A variety of substitutions and/or variations are contemplated that allow variability in the degree of modulation of sodium channels. The following substitutions and/or variations are thus intended to be merely exemplary of embodiments of this invention, and should not be seen as limiting. Table 1, for example, shows non-limiting examples of peptide analogs obtained in the context of this invention to demonstrate a few of the contemplated moieties. C₂₄ from SEQ ID 001 is a free-thiol substituted cysteine in some embodiments. C₂₄ is replaced by an alternative amino acid residue in other embodiments.

In other embodiments the C₂₄ residue of SEQ ID 001 forms a dimer with a variety of useful peptides. In one aspect, for example, the dimer can be a second peptide according to SEQ ID 001, as is shown in Table 1 as SEQ ID 015. It is noted that the second peptide can have the exact sequence of SEQ ID 001, a substantially similar sequence at to SEQ ID 001, or any degree of modification that allows beneficial functionality of the peptide.

In other embodiments, C₂₄ is reversibly modified with a molecule through a disulfide linkage. Numerous disulfide linkages are known, and any such linkage that can be utilized that allows sufficient functionality of the peptide is considered to be within the present scope. Non-limiting examples of such substitution molecules can include glutathione, cysteine, cysteamine, DTNB, selenocysteine, selenoglutathione, and any product of a reaction of C₂₄ with an alkanethiosulfonate reagent or a thiosulfate reagent, and combinations thereof. A few examples from Table 1 showing reversible substitutions include SEQ ID 003, SEQ ID 008, SEQ ID 009, SEQ ID 011, SEQ ID 012, SEQ ID 013, SEQ ID 015, SEQ ID 019, SEQ ID 020, and SEQ ID 021.

In other aspects, C₂₄ is irreversibly substituted with a molecule. Numerous irreversible substitutions are contemplated, and any such substitution that allows sufficient functionality of the peptide is considered to be within the present scope. Non-liming examples of irreversibly substituted molecules include acetamidomethyl, products of a reaction of C₂₄ with maleimides, vinyl sulfones and related α,β-unsaturated systems, β-haloethylamine, α-halocarbonyls, or a combination thereof. On example from Table 1 showing irreversible substitutions is SEQ ID 014.

In another aspect, a peptide is provided having a sequence of SEQ ID 001, wherein X₂₃ is aspartic acid, C₂₄ is an un-substituted cysteine, and X₂₅ is tyrosine, where such a sequence is GWCGDOGATC GKLRLYCCSG FCDCYTKTC-X₃₀̂ (SEQ ID 022). In a more specific aspect, X₃₀ can be SEQ ID 002, where the resulting peptide would be GWCGDOGATC GKLRLYCCSG FCDCYTKTCK DKSSA (SEQ ID 023).

In a further aspect, a peptide is provided having a sequence of SEQ ID 001, wherein X₂₃ is aspartic acid, C₂₄ is substituted with cystamine, and X₂₅ is tyrosine. In a more specific aspect, X₃₀ can be SEQ ID 002, where the resulting peptide can have a sequence of SEQ ID 011.

It is also noted that in some aspects, a peptide according to aspects of the present invention can further include a label, such as, for example, a fluorescent label. Such a labeled peptide can be used to probe libraries, such as small molecule libraries.

TABLE 1

| | | | rNaᵥ 1.7 |
|---|---|---|---|
| | | hNa_{V} 1.7 | % block |
| | | IC₅₀ or | peptide |
| Peptide | Sequence | % block | concentration |
| C.geo1[1-35] SEQ ID 003 | 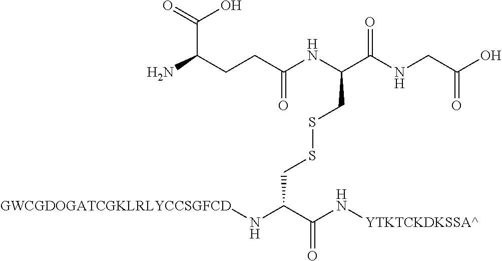 | 1.4 μM | 70% |
| C.geo1[C24Abu] SEQ ID 004 | 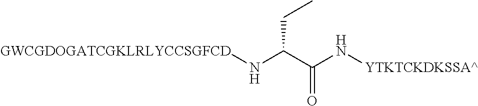 | >10 μM | 20% (33 μM) |
| C.geo1[C24S] SEQ ID 005 | 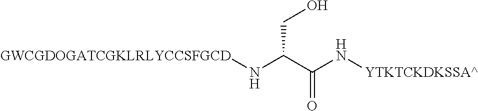 | >10 μM | 20% (33 μM) |
| C.geo1[C24K] SEQ ID 006 | 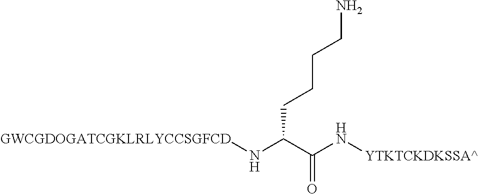 | >10 μM | 20% (33 μM) |
| C.geo1[C24E] SEQ ID 007 | 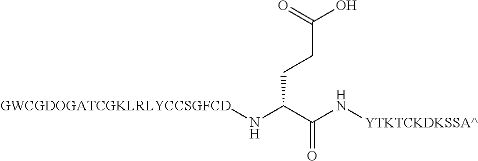 | >10 μM | 15% (33 μM) |
| C.geeo1[K27G] SEQ ID 008 | 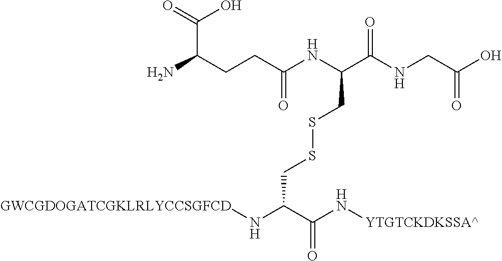 | 1.6 μM | 60% (33 μM) |
| C.geo1[O6A] SEQ ID 009 | 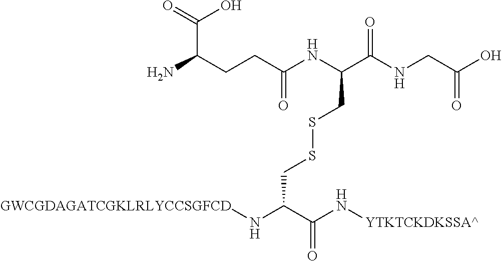 | >10 μM | 60% (33 μM) |
| C.geo1[desGSH] SEQ ID 010 | 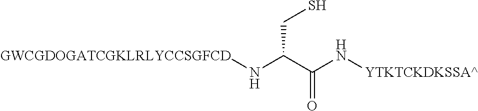 | 71 nM | 70% |
| C.geo1[cystamine] SEQ ID 011 | 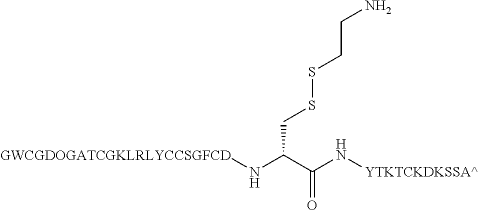 | 72 nM | 70% |
| C.geo1[cystine] SEQ ID 012 | 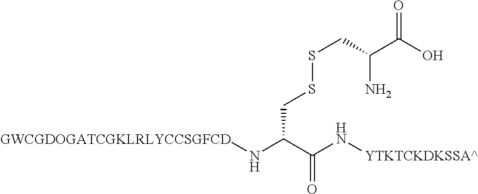 | 925.8 nM | not tested |
| C.geo1[DTNB] SEQ ID 013 | 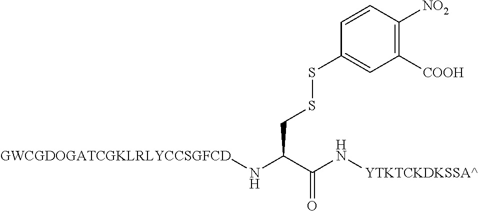 | >3 μM | not tested |
| C.geo1[C24Cys(Acm)] SEQ ID 014 | 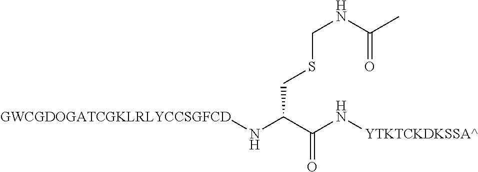 | >1 uM | 70% (33 μM) |
| C.geo1[dimer] SEQ ID 015 | 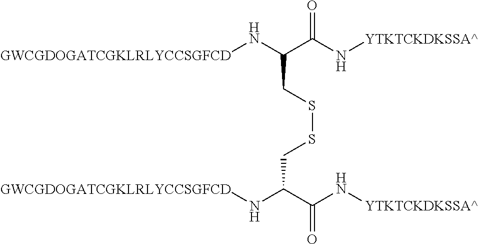 | 437 nM | 70% (30 μM) |
| C.geo1[C24D-Cys] SEQ ID 016 | 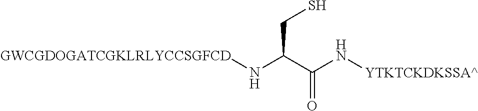 | 2.86 μM | not tested |
| C.geo1[C24HoCys] SEQ ID 017 | 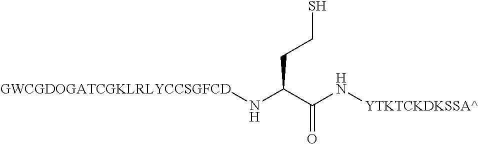 | 1.5 μM | not tested |
| C.geo1[C24Pen] SEQ ID 018 | 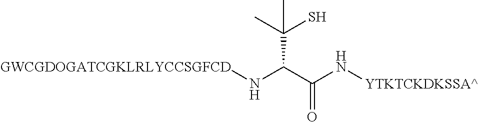 | >1 μM | not tested |
| C.geo1[D23Gla; cystamine] SEQ ID 019 | 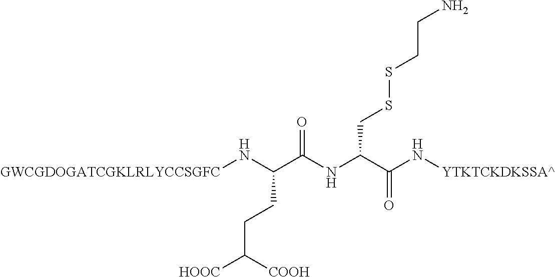 | 77.9% block at 3 μM | not tested |
| C.geo1[D23N; cystamine] SEQ ID 020 | 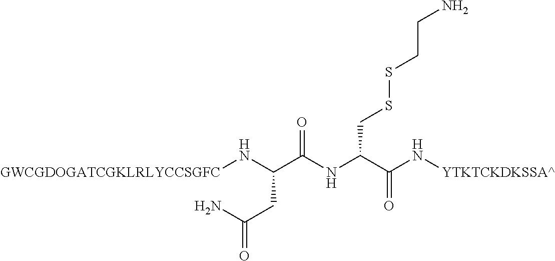 | 23.8% block at 300 nM | not tested |
| C.geo1[Y25D; cystamine] SEQ ID 021 | 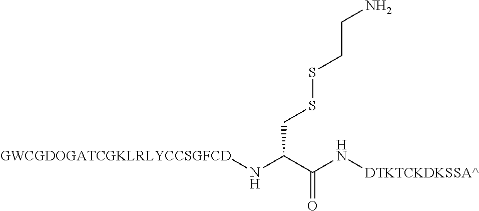 | 16.2% block at 300 nM | not tested |

It is noted that a variety of oxidative folding methods can be utilized to generate peptide analogs, and that any useful folding technique is considered to be within the present scope. Various folding methods utilized to generate the exemplary peptides of Table 1 can be as follows: folding in the presence of a 1:1 mixture of GSSH:GSH can be used to generate SEQ IDs 003-009 and SEQ ID 014; folding in the presence of cystamine can be used to generate SEQ ID 011 and SEQ IDs 019-021; folding in the presence of cystine can be used to generate SEQ ID 012; and folding in the presence of copper ions can be used to generate SEQ ID 010 and SEQ IDs 016-018. As other examples, SEQ ID 015 and SEQ ID 013 can be prepared from SEQ ID 010 by reacting it with DMSO and Ellman's reagent (DTNB) respectively. Peptides can subsequently be purified by, for example, RP HPLC, and masses can be confirmed by MALDI mass spectrometry.

In another aspect of the present invention, a peptide is provided having a sequence of DWCGDAGDAC GTLKLRCCSG LCNQYSGTCTĜ (SEQ ID 24), where ̂ is a carboxylated C-terminus. In yet another aspect, a peptide is provided having a sequence of CVGRDSKCGP PPCCMGMTCN YERVRKCT̂ (SEQ ID 25), where ̂ is a carboxylated C-terminus.

Table 2 shows a selectivity profile for various active peptide analogs against subtypes of hNa_{V}1s given as IC₅₀ data. The data in this Table show that all three peptides are potent inhibitors of hNaᵥ1.7. They also showed similarity in hNaᵥ1.7 potency between C. geo1[desGSH] (SEQ ID 010) and C. geo1[cystamine] (SEQ ID 011), which indicated that the second analog could be used as a substitute for the less stable C. geo1[desGSH] (SEQ ID 003). These data reveal that analogs did not block TTX-resistant hNa_{V}1.5.

TABLE 2

| | C.geo1[1-35] | C.geo1[desGSH] | C.geo1 [cystamine] |
|---|---|---|---|
| hNaᵥ | SEQ ID 003 | SEQ ID 010 | SEQ ID 011 |
| 1.1 | 760 | 28 | 89 |
| 1.2 | 1110 | 52 | 51 |
| 1.3 | >10000 | 126 | 336 |
| 1.4 | 1091 | 14 | 14 |
| 1.5 | >10000 | >10000 | >10000 |
| 1.6 | 757 | 21 | 89 |
| 1.7 | 1396 | 71 | 72 |

It is noted that many amino acids in a given peptide can be variable, and such variations are considered within the present scope. For example, Pro residues may be substituted with hydroxy-Pro; hydroxy-Pro residues may be substituted with Pro residues; Arg residues may be substituted by Lys, ornithine, homoargine, nor-Lys, N-methyl-Lys, N,N-dimethyl-Lys, N,N,N-trimethyl-Lys or any synthetic basic amino acid; Lys residues may be substituted by Arg, ornithine, homoargine, nor-Lys, or any synthetic basic amino acid; Tyr residues may be substituted with any synthetic hydroxy containing amino acid; Ser residues may be substituted with Thr or any synthetic hydroxylated amino acid; Thr residues may be substituted with Ser or any synthetic hydroxylated amino acid; Phe and Trp residues may be substituted with any synthetic aromatic amino acid; and Asn, Ser, Thr or Hyp residues may be glycosylated. Tyr residues may also be substituted with the 3-hydroxyl or 2-hydroxyl isomers (meta-Tyr or ortho-Tyr, respectively) and corresponding O-sulpho- and O-phospho-derivatives or may be substituted with nor-Tyr, nitro-Tyr, mono-iodo-Tyr or di-iodo-Tyr. Aliphatic amino acids may be substituted by synthetic derivatives bearing non-natural aliphatic branched or linear side chains CₙH₂ₙ₊₂ up to and including n=8. Leu residues may be substituted with Leu(D). Trp residues may be substituted with halo-Trp, Trp(D) or halo-Trp(D). The halogen is iodo, chloro, fluoro or bromo; preferably iodo for halogen substituted-Tyr and bromo for halogen-substituted Trp. In addition, the halogen can be radiolabeled, e.g., ¹²⁵I-Tyr.

Examples of synthetic aromatic amino acids include, but are not limited to, nitro-Phe, 4-substituted-Phe wherein the substituent is C₁-C₃ alkyl, carboxyl, hyrdroxymethyl, sulphomethyl, halo, phenyl, —CHO, —CN, —SO₃H and —NHAc. Examples of synthetic hydroxy containing amino acids, include, but are not limited to, 4-hydroxymethyl-Phe, 4-hydroxyphenyl-Gly, 2,6-dimethyl-Tyr and 5-amino-Tyr. Examples of synthetic basic amino acids include, but are not limited to, N-1-(2-pyrazolinyl)-Arg, 2-(4-piperinyl)-Gly, 2-(4-piperinyl)-Ala, 2-[3-(2S)pyrrolininyl)-Gly and 2-[3-(2S)pyrrolininyl)-Ala. These and other synthetic basic amino acids, synthetic hydroxy containing amino acids or synthetic aromatic amino acids are described in Building Block Index, Version 3.0 (1999 Catalog, pages 4-47 for hydroxy containing amino acids and aromatic amino acids and pages 66-87 for basic amino acids; see also the website “amino-acids dot com”), incorporated herein by reference, by and available from RSP Amino Acid Analogues, Inc., Worcester, Mass.

In other aspects, Asn residues may be modified to contain an N-glycan and the Ser, Thr and Hyp residues may be modified to contain an O-glycan (e.g., g-N, g-S, g-T and g-Hyp). A glycan can refer to any N-, S- or O-linked mono-, di-, tri-, poly- or oligosaccharide that can be attached to any hydroxy, amino or thiol group of natural or modified amino acids by synthetic or enzymatic methodologies known in the art. The monosaccharides making up the glycan can include D-allose, D-altrose, D-glucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose. These saccharides may be structurally modified, e.g., with one or more O-sulfate, O-phosphate, O-acetyl or acidic groups, such as sialic acid, including combinations thereof. The glycan may also include similar polyhydroxy groups, such as D-penicillamine 2,5 and halogenated derivatives thereof or polypropylene glycol derivatives. The glycosidic linkage is β and 1-4 or 1-3, preferably 1-3. The linkage between the glycan and the amino acid may be α or β, preferably α and is 1-.

Mucin type O-linked oligosaccharides are attached to Ser or Thr (or other hydroxylated residues of the present peptides) by a GalNAc residue. The monosaccharide building blocks and the linkage attached to this first GalNAc residue define the “core glycans,” of which eight have been identified. The type of glycosidic linkage (orientation and connectivities) are defined for each core glycan. Suitable glycans and glycan analogs are described further in U.S. Pat. No. 6,369,193 and in International Publication No. WO 00/23092, each incorporated herein by reference. In one aspect, a glycan can be Gal(β1→3)GalNAc(α1→).

The present peptides can be pharmacologically beneficial because they exhibit activity in animals, for example, in Nav1.7 channel blocking or inhibition. As such, compounds incorporating such peptides can be of use in the treatment of disorders for which a blocker or inhibitor for sodium channels (e.g. Nav1.7) is indicated.

In one aspect, pharmaceutical compositions are contemplated including a peptide having at least 95% sequence identity to SEQ ID 001, including pharmaceutically acceptable salts or solvates thereof, in a pharmaceutically acceptable carrier. In another aspect, the peptide can have a sequence of SEQ ID 001. In yet another aspect, X₂₃ can be aspartic acid, C₂₄ can be an un-substituted cysteine, and X₂₅ can be tyrosine. In a further aspect, X₃₀ can be SEQ ID 002. Additionally, in another aspect, X₂₃ can be aspartic acid, C₂₄ can be substituted with cystamine, and X₂₅ can be tyrosine. In a further aspect, X₃₀ can be SEQ ID 002.

Pharmaceutical compositions containing a compound, such as a peptide as an active ingredient can be prepared according to conventional pharmaceutical compounding techniques. See, for example, Remington: The Science and Practice of Pharmacy, 21st Ed., Lippincott Williams & Wilkins, Philadelphia, 2005. Typically, an therapeutically effective amount of active ingredient can be admixed with a pharmaceutically acceptable carrier. The carrier can take a wide variety of forms depending on the form of preparation desired for administration, e.g., intravenous, oral, parenteral or intrathecally. For examples of delivery methods see U.S. Pat. No. 5,844,077, incorporated herein by reference.

For oral administration, compound can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier.

For parenteral administration, compounds can be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

A variety of administration routes are available. The particular mode selected will depend of course, upon the particular drug selected, the severity of the condition being treated and the dosage required for therapeutic efficacy. The methods of this disclosure, generally speaking, can be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, epidural, irrigation, intramuscular, release pumps, or infusion. For example, administration of the active agent according to this invention may be achieved using any suitable delivery means, including those described in U.S. Pat. No. 5,844,077, incorporated herein by reference.

Alternatively, targeting therapies can be used to deliver the peptide composition more specifically to certain types of cell, by the use of targeting systems such as antibodies or cell specific ligands. Targeting may be desirable for a variety of reasons, e.g. if the agent is unacceptably toxic, or if it would otherwise require too high a dosage, or if it would not otherwise be able to enter the target cells.

The active agents, which are peptides, can also be administered in a cell based delivery system in which a DNA sequence encoding an active agent is introduced into cells designed for implantation in the body of the patient, especially in the spinal cord region. Suitable delivery systems are described in U.S. Pat. No. 5,550,050 and published PCT Application Nos. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of the developed sequences and the known genetic code.

In some aspects, an active agent can be administered in a therapeutically effective amount. A “therapeutically effective amount” or simply “effective amount” of an active compound refers to a sufficient amount of the compound to treat the desired condition at a reasonable benefit/risk ratio applicable to any medical treatment. The actual amount administered, and the rate and time-course of administration, may depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy.

Dosage can be adjusted appropriately to achieve desired drug levels, locally or systemically. Typically the active agents of the present disclosure exhibit their effect at a dosage range from about 0.001 mg/kg to about 250 mg/kg, preferably from about 0.01 mg/kg to about 100 mg/kg of the active ingredient, more preferably from about 0.05 mg/kg to about 75 mg/kg. A suitable dose can be administered in multiple sub-doses per day. Typically, a dose or sub-dose may contain from about 0.1 mg to about 500 mg of the active ingredient per unit dosage form. Another dosage can contain from about 0.5 mg to about 100 mg of active ingredient per unit dosage form. Dosages are generally initiated at lower levels and increased until desired effects are achieved. In the event that the response in a subject is insufficient at such doses, even higher doses (or effective higher doses by a different, more localized delivery route) may be employed to the extent that patient tolerance permits. Continuous dosing over, for example, 24 hours or multiple doses per day are contemplated to achieve appropriate systemic levels of compounds.

Advantageously, the compositions are formulated as dosage units, each unit being adapted to supply a fixed dose of active ingredients. Tablets, coated tablets, capsules, ampoules and suppositories are examples of dosage forms according to the invention.

It is noted that exact individual dosages, as well as daily dosages, can be determined according to standard medical principles under the direction of a physician or veterinarian for use humans or animals.

The pharmaceutical compositions will generally contain from about 0.0001 to 99 wt. %, or about 0.001 to 50 wt. %, or about 0.01 to 10 wt. % of the active ingredient by weight of the total composition. In addition to the active peptide, the pharmaceutical compositions and medicaments can also contain other pharmaceutically active compounds. Examples of other pharmaceutically active compounds include, but are not limited to, analgesic agents, cytokines and therapeutic agents in all of the major areas of clinical medicine. When used with other pharmaceutically active compounds, the peptides of the present invention may be delivered in the form of drug cocktails. A cocktail is a mixture of any one of the compounds useful with this invention with another drug or agent. In this embodiment, a common administration vehicle (e.g., pill, tablet, implant, pump, injectable solution, etc.) would contain both the instant composition in combination with a supplementary potentiating agent. The individual drugs of the cocktail are each administered in therapeutically effective amounts. A therapeutically effective amount will be determined by the parameters described above; but, in any event, is that amount which establishes a level of the drugs in the area of body where the drugs are required for a period of time which is effective in attaining the desired effects.

A Nav1.7 blocker or inhibitor can thus be usefully combined with another pharmacologically active compound, or with two or more other pharmacologically active compounds, particularly in the treatment of pain. Such combinations offer the possibility of significant advantages, including patient compliance, ease of dosing and synergistic activity. In such combinations, a conopeptide described herein can be administered simultaneously, sequentially or separately in combination with the other therapeutic agent or agents. Agents which may be administered with a conopeptide described herein include agents described in US 2012/0010207, which is incorporated herein by reference.

The term “pharmaceutical composition” refers to physically discrete coherent portions suitable for medical administration. “Pharmaceutical composition in dosage unit form” refers to physically discrete coherent units suitable for medical administration, each containing a daily dose or a multiple (up to four times) or a sub-multiple (down to a fortieth) of a daily dose of the active compound in association with a carrier and/or enclosed within an envelope. Whether the composition contains a daily dose, or for example, a half, a third or a quarter of a daily dose, will depend on whether the pharmaceutical composition is to be administered once or, for example, twice, three times or four times a day, respectively.

The term “salt”, as used herein, denotes acidic and/or basic salts, formed with inorganic or organic acids and/or bases, preferably basic salts. While pharmaceutically acceptable salts are preferred, particularly when employing the compounds of the invention as medicaments, other salts find utility, for example, in processing these compounds, or where non-medicament-type uses are contemplated. Salts of these compounds may be prepared by art-recognized techniques.

Examples of such pharmaceutically acceptable salts include, but are not limited to, inorganic and organic addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, alpha tocopherol and the like; and the metal chelating agents such as citric acid, ethylenediamine tetraacetic acid (EDTA), sorbitol, tartaric acid, phosphoric acid and the like.

Sodium channels such as Nav1.7 may play a role in various pain states, including acute, inflammatory and/or neuropathic pain. Deletion of the SCN9A gene in nociceptive neurons of mice led to a reduction in mechanical and thermal pain thresholds and reduction or abolition of inflammatory pain responses. In humans, Nav1.7 protein has been shown to accumulate in neuromas, particularly painful neuromas. Gain of function mutations of Nav1.7, both familial and sporadic, have been linked to primary erythermalgia, a disease characterized by burning pain and inflammation of the extremities, and paroxysmal extreme pain disorder. Further, non-selective sodium channel blockers lidocaine and mexiletine can provide symptomatic relief in cases of familial erythermalgia and carbamazepine is effective in reducing the number and severity of attacks in PEPD. Further evidence of the role of Nav1.7 in pain is found in the phenotype of loss of function mutations of the SCN9A gene.

As such, in another aspect of the present disclosure, a method of treating a condition or treating effects of a condition in a subject where sodium channels exhibit increased activity is provided. Such a method can include administering to the subject a therapeutically effective amount of a composition as has been described herein to modulate the activity of the sodium channels. Non-limiting examples of such conditions can include, acute pain, chronic pain, neuropathic pain, cancer pain, diabetic neuropathy, inflammatory pain, trigeminal pain, perioperative pain, visceral pain, nociceptive pain including post-surgical pain, and mixed pain types involving the viscera, gastrointestinal tract, cranial structures, musculoskeletal system, spine, urogenital system, cardiovascular system and CNS, including cancer pain, back and orofacial pain, or a combination thereof. It is also contemplated that such a condition can be a neurological condition, including spinal cord injury, traumatic brain injury, peripheral nerve injury, and the like.

Peptides of the invention can be tested for their effect in reducing or alleviating pain using animal models, such as the SNL (spinal nerve ligation) rat model of neuropathic pain, carageenan induced hyperalgesia model, the Freund's complete adjuvant (CFA)-induced hyperalgesia model, the thermal injury model, the formalin model and the Bennett Model and other modes as described in U.S. Pat. Appl. No. 2011/0124711A1 and U.S. Pat. No. 7,998,980. Carageenan induced hyperalgesia and (CFA)-induced hyperalgesia are models of inflammatory pain. The Bennett model provides an animal model for chronic pain.

Any of the foregoing animal models may be used to evaluate the efficacy of peptides of the invention in treating pain. The efficacy can be compared to a no treatment or placebo control. Additionally or alternatively, efficacy can be evaluated in comparison to one or more known pain relieving medicaments.

Generally, physiological pain is an important protective mechanism designed to warn a subject of danger from potentially injurious stimuli. The pain system operates through a specific set of primary sensory neurons, and in some cases is activated by noxious stimuli via peripheral transducing mechanisms. These sensory fibers are known in the art as nociceptors, and they are characteristically small diameter axons with slow conduction velocities. Nociceptors can encode the intensity, duration, and quality of noxious stimuli; topographical organization of nociceptor projections to the spinal cord also allows stimuli location to be encoded.

Nociceptors are found on nociceptive nerve fibers of which there are two main types, A-delta fibers (myelinated) and C fibers (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitization in the periphery, locally around the injury and centrally where the nociceptors terminate. These effects lead to a heightened sensation of pain. In acute pain these mechanisms can be useful, in promoting protective behaviors which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibers associated with maladaptation and aberrant activity.

Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994). The activation of nociceptors activates two types of afferent nerve fibers. Myelinated A-delta fibers transmit rapidly and are responsible for sharp and stabbing pain sensations, whilst unmyelinated C fibers transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumor related pain (e.g. bone pain, headache, facial pain or visceral pain) or pain associated with cancer therapy (e.g. post-chemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertebral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life. The symptoms of neuropathic pain are difficult to treat, as they are often heterogeneous even between patients with the same disease. They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain. Arthritic pain is the most common inflammatory pain. Rheumatoid disease is one of the commonest chronic inflammatory conditions in developed countries and rheumatoid arthritis is a common cause of disability. The exact aetiology of rheumatoid arthritis is unknown, but current hypotheses suggest that both genetic and microbiological factors may be important. It has been estimated that almost 16 million Americans have symptomatic osteoarthritis (OA) or degenerative joint disease, most of who are over 60 years of age, and this is expected to increase to 40 million as the age of the population increases, making this a public health problem of enormous magnitude. Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain includes functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhea, cystitis and pancreatitis and pelvic pain.

It should be noted that some types of pain have multiple aetiologies and thus can be classified in more than one area, e.g. back pain and cancer pain have both nociceptive and neuropathic components. Other types of pain include: (a) pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis; (b) heart and vascular pain, including pain caused by angina, myocardical infarction, mitral stenosis, pericarditis, Raynaud's phenomenon, scleredoma and skeletal muscle ischemia; (c) head pain, such as migraine (including migraine with aura and migraine without aura), cluster headache, tension-type headache mixed headache and headache associated with vascular disorders; (d) erythermalgia; and (e) orofacial pain, including dental pain, otic pain, burning mouth syndrome and temporomandibular myofascial pain.

### EXAMPLES

### Example 1

### Venom Screening

Material from 10 Conus species has been extracted, fractionated, and screened for block of hNaV1.7 using the QPatch assay. A summary of Conus species and fractionation data is provided in Table 1. Based on initial efforts, a total of 393 fractions have been collected and screened for activity. Of these initial crude fractions, 29 fractions were identified as ‘hits’, exhibiting ≧30% block of hNaV1.7 (˜9.2% of fractions were found to be active) (Table 3).

TABLE 3
Overview of Screening Venom Libraries

| | Fractionation Number of ‘hits’ |
|---|---|
| Species | (block ≧ 30%) |
| C. miles | 2 |
| C. vexillum | 2 |
| C. geographus | 11 |
| C. betulinus | 0 |
| C. textile | 2 |
| C. striatus | 9 |
| C. magus | 0 |
| C. marmoreus | 1 |
| C. distans | 1 |
| C. quercinas | 1 |

### Example 2

### Screening of Conpeptide Fractions

From screening and deconvolution of venom fractions, we identified Conus geographus as one promising species in possessing conopeptide components that block hNaV1.7. Initial screening results of C. geographus venom are summarized in Table 4.

TABLE 4
Initial QPatch Results From the Crude Fractionation of C. geographus.

| Fraction | 1 | 2 | 3 | 4 | 5 | 6 | 7 | 8 | 9 | 10 |
|---|---|---|---|---|---|---|---|---|---|---|
| % Inh. | 24^{a} | 32 | 21 | 42 | 27 | 37 | 28 | 41^{a} | 23 | 59^{a} |
| Fraction | 11 | 12 | 13 | 14 | 15 | 16 | 17 | 18 | 19 | 20 |
| % Inh. | 11 | 31 | 15 | 6 | 22 | 8 | 21 | 2 | 10^{a} | 15 |
| Fraction | 21 | 22 | 23 | 24 | 25 | 26 | 27 | 28 | 29 | 30 |
| % Inh. | 18 | 26 | 5 | 5 | 9 | 5 | 9 | 15 | 10 | 10 |
| Fraction | 31 | 32 | 33 | 34 | 34 | 36 | 37 | 38 | 39 | 40 |
| % Inh. | −14 | 20 | 42 | 27 | 6 | 3 | −1 | 7 | 18 | 13 |
| Fraction | 41 | 42 | 43 | 44 | 45 | 46 | 47 | 48 | 49 | 50 |
| % Inh. | 3 | 2 | 20 | 8 | 37 | 40 | 28 | 24^{b} | 33^{a} | 41^{a} |

| Fraction | 51 | 52 | 53 | 54 | 55 |
|---|---|---|---|---|---|
| % Inh. | 17^{a} | 22 | −1 | 5 | −7 |

Conopeptide material extracted from approximately 600 mg lyophilized C. geographus ducts and was screened against hNaV1.7. Fraction amounts corresponding to approximately 6 mg equivalents of total conopeptide material were re-suspended in 200 μL volume.
^{a}denotes shorter exposure due to seal breakdown.
^{b}n = 2
Fractions exhibiting ≧30% block of hNaV1.7 are indicated in bold

### Example 3

### Deconvolution and Identification of Hits

Initial screening of C. geographus crude fractions revealed two major groupings of fractions that blocked the hNaV1.7 response (See Table 4). Further purification of these fractions resulted in sub-fractions that exhibited hNaV1.7 block greater than 30%: SubFr 34.4 (69%), SubFr 34.5 (69% block), SubFr 33.5 (34% block), SubFr 33.6 (40% block), SubFr 33.7 (31% block) (Table 5).

TABLE 5
QPatch Results From Sub-fractionation of C. geographus Fractions

| Fraction | 32 | 32.2 | 32.3 | 32.4 | 32.5 | 32.6 | 32.7 |
|---|---|---|---|---|---|---|---|
| % Inh. | 20 | 15 | 18 | 18 | 25 | 26 | 29 |
| Fraction | 33 | 33.3 | 33.4 | 33.5 | 33.6 | 33.7 | 33.8 |
| % Inh. | 42 | 31 | 23 | 34 | 40 | 31 | 20 |

| Fraction | 34 | 34.3 | 34.4 | 34.5 | 34.6 |
|---|---|---|---|---|---|
| Inh. | 27 | 33 | 69 | 69 | 18 |
| Fraction | 45 | 45.4 | 45.5 | 45.6 | 45.7 |
| % Inh. | 37 | 22^{a} | 13 | 30 | 8 |

| Fraction | 46 | 46.3 | 46.4 |
|---|---|---|---|
| % Inh. | 40 | 11 | 22 |
| Fraction | 47 | 47.4 | 47.5 |
| % Inh. | 28 | 23 | 23 |

Fractions exhibiting ≧30% block of hNaV1.7 are indicated in bold

### Example 4

### Characterization of the Nav1.7 Active Peptides from C. Geographus

Initial sequencing efforts of the C. geographus active peptide identified in SubFr 33.6 revealed an incomplete peptide sequence (GXCCGDOGATC KLRLYCCSGF CDCYTcTc . . . ) where X denotes ambiguity in the amino acid sequence SEQ ID 026. To elucidate the complete sequence of this peptide, both mass spectrometry methods and molecular biology techniques were employed in parallel.

Molecular Biology Methods. Due to limited quantities of the native active peptide, RACE-PCR experiments were conducted in an attempt to elucidate the entire peptide sequence. From PCR experiments, the entire sequence was identified

SEQ ID 027
(GWCGDPGATC GKLRLYCCSG FCDCYTKTCK DKSSA).

Furthermore, transcriptome information confirmed this sequence in multiple locations using RNA isolated from C. geographus ducts.

Mass Spectrometry Analysis. The calculated mass (3739.2 Da), based upon the sequence obtained from PCR experiments, and the experimentally-determined mass (3934.4 Da) differed by 195.3 Da suggesting the presence of modified residues within the sequence.

Solid Phase Peptide Synthesis. Based on the unmodified sequence obtained from the PCR and transcriptome data, analogs of the C. geographus peptide were designed and synthesized by SPPS using standard Fmoc-protocols. Initial syntheses lacked Ser-34 (below). Synthesis of the active peptide was repeated successfully resulting in analogs C. geo1[1-35] (SEQ ID 003) and C. geo1[C24Abu] (SEQ ID 004).

C. geo1[des-Ser34]:
SEQ ID 028
GWCGDOGATCGKLRLYCCSGFCDCYTKTCKDKS_A\hat{ }
C. geo1[C24Abu,des-Ser34]:
SEQ ID 029
GWCGDOGATCGKLRLYCCSGFCD(Abu)YTKTCKDKS_A\hat{ }
C. geo1[1-35]:
SEQ ID 003
GWCGDOGATCGKLRLYCCSGFCDCYTKTCKDKSSA\hat{ }
C. geo1[C24Abu]:
SEQ ID 004
GWCGDOGATCGKLRLYCCSGFCD(Abu)YTKTCKDKSSA\hat{ }
*Note:
Abu = Fmoc-aminobutyric acid;
\hat{ }denotes carboxylated C-terminus

Synthetic peptides were folded using both air oxidation and glutathione-assisted oxidation methods. Folding mixtures were purified by semi-preparative RP-HPLC and the molecular masses of the folding products were confirmed by MALDI-TOF mass spec.

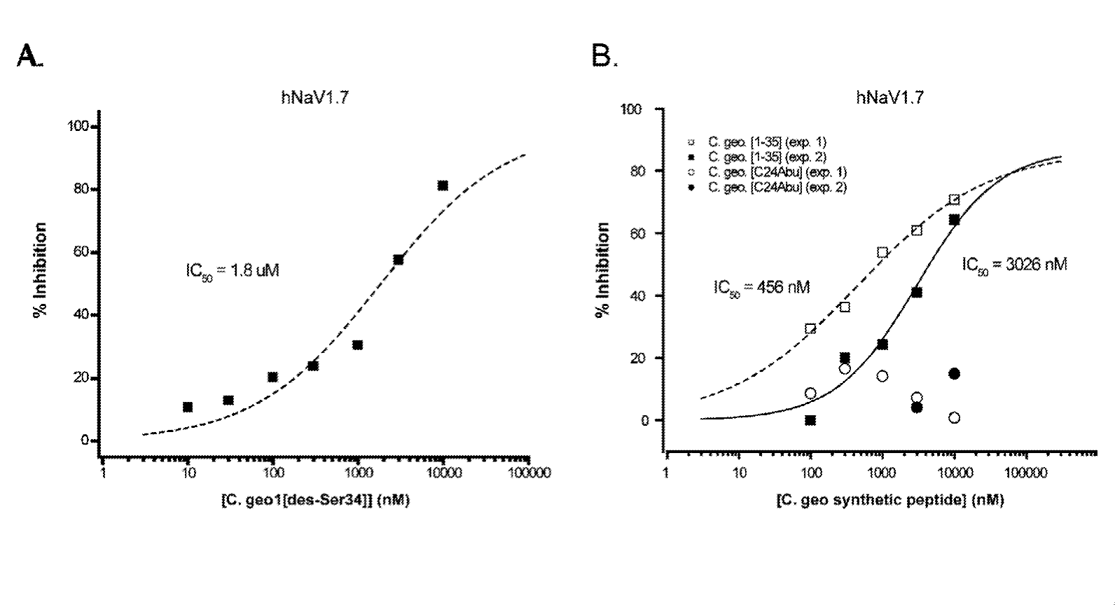

Electrophysiology. Folded peptide analogs were first tested for activity at the University of Utah against NaV1.7 from rat. C. geo1[des-Ser34] (SEQ ID 028) exhibited very slow reversibility and resulted in 70% block using 3.3 μM peptide. Isosteric replacement of Cys24 with aminobutyric acid (Abu) in C. geo1[C24Abu,des-Ser34] (SEQ ID 004) decreased NaV1.7 block to 20% at 10 μM and was quickly reversible (data not shown). These data suggest that Cys24 is integral for efficient block of NaV1.7. As such, 10 nmols of C. geo1[des-Ser34] (SEQ ID 028) was subsequently used for testing against human NaV1.7 in the QPatch assay (FIG. 1).

RACE-PCR: RACE-PCR was employed to capture the entire sequence (unmodified; SEQ ID 030):

GGTQHRALRS TIKLSLLRQH RGWCGDPGAT
CGKLRLYCCS GFCDCYTKTC KDKSSASSPS VLYPFLPES.

Δmass between unmodified sequence and MALDI-ToF data was +197.1 Da suggesting modification of the sequence.

MALDI-ToF analysis: MALDI-ToF analysis of C. geo[1-35, des-Ser34] (SEQ ID 028) and C. geo1[1-35] (SEQ ID 003) showed the peptide to be ‘heavy’ by 305 Da indicating peptide-GSH adduct formed at Cys-24. Peptide-adducts may suggest bulky modification of Cys-24, e.g. S-linked glycosylation.

### Example 5

### Verification of which Cys (Cys22 or Cys24) is the Free Cys Residue in Synthetic, Folded C. Geo1

The free cysteine of folded C. geo1[desGSH] (SEQ ID 010) was alkylated with 4-vinylpyridine (VP) and then the peptide was reduced and all remaining cysteines were alkylated with iodoacetamide (IAM-iodoacetamide). Peptide treated this way was then digested with Endoproteinase AspN, subjected to analytical reversed phase (RP) HPLC, and all products were collected and analyzed by MALDI-TOF. The mass of peak 1 (17.16 min, analytical HPLC; [M+H]+=1123.56) was found to be the same as expected mass ([M+H]+=1123.93) for a peptide fragment DC(VP)YTKTC(IAM)K (SEQ ID 031) of digested C. geo1. The results show that the Cys24 is the one with a free thiol and likely (disulfide) linked to GSH in synthetic C. geo1.

### Example 6

### Connectivity of Cys Residues in Synthetic C. Geo1

For this example C. geo1[desGSH] (SEQ ID 010) was used. The peptide was treated with 4-vinylpyridine and purified by HPLC. Next, it was treated with tris(2-carboxyethyl)phosphine (TCEP) for 45 min, which caused partial reduction of the peptide. Finally, the mixture was treated with N-ethylmaleimide (NEM), and purified by analytical RP-HPLC. Masses of collected peaks 1 through 5 were analyzed by MALDI-TOF. Following results were obtained:
a) Peak 1 [M+H]⁺_{found}=3842.37, which corresponds to 3 disulfide closed and alkylated Cys²⁴;
b) Peak 2 [M+H]⁺_{found}=4093.56, 2 disulfide bridges closed, 1 disulfide alkylated with NEM;
c) Peak 3 [M+H]⁺_{found}=4345.69, 1 disulfide bridge closed, 2 disulfide alkylated with NEM;
d) Peak 4 [M+H]⁺_{found}=4345.66, 1 disulfide bridge closed, 2 disulfide alkylated with NEM;
e) Peak 5 [M+H]⁺_{found}=4598.60, 3 disulfide bonds alkylated with NEM.
   Intermediates labeled as Peak 1, 2 and 3 were treated with TCEP for 1 h and then reacted with IAM. The resulting material was purified by RP-HPLC and then treated with modified trypsin for 3 h. This material was next analyzed by MALDI-TOF. Based on the overall data, it was determined that the connectivity in synthetic C. geo1[desGSH] (SEQ ID 010) is: Cys3-Cys18, Cys10-Cys22 and Cys17-Cys29, which falls into a predicted VI/VII cysteine framework. It was also an additional confirmation that Cys24 was not involved in disulfide-bond formation but nevertheless involved in the functional activity of the synthetic C. geo1.

### Example 7

### Discovery of C. Geo2

In addition to the biologically-active C. geo1 peptide isolated from Conus geographus, a second active peptide has been identified from sub-fraction 34.5 (C. geo2). QPatch assay of the isolated peptide resulted in 69% block of hNaV1.7. The isolated native peptide was reduced and alkylated by treatment with dithiothreitol and 4-vinylpyridine in preparation for sequencing by Edman degradation at the University of Utah. Sequencing efforts revealed the partial peptide sequence of XXCGDAGDA CGTLKLRCCS GLCNQYSGTC S . . . , (SEQ ID 032) where X denotes ambiguity in the amino acid sequence. Using the partial sequence, the complete peptide sequence was retrieved by searching C. geographus transcriptome data as described previously. The complete sequence of C. geo2 exhibits the canonical ω-conopeptide cysteine framework and shares a fair amount of sequence identity with C. geo1 (˜55% homologous); however, C. geo2 lacks the additional cysteine (Cys-24) observed in C. geo1 (See alignment below).

C. geo1
(SEQ ID 003)
GWCGDOGATCGKLRLYCCSGFCDCYTKTCKDKSSA\hat{ }
C. geo2
(SEQ ID 024)
DWCGDAGDACGTLKLRCCSGLCNQYSGTCTG\hat{ }
*Note:
Bold represents homology between sequences;
\hat{ }denotes carboxylated C-terminus

Of particular interest is that members of the ω-conopeptide family typically possess a C-terminal [Ser-Ser-Ala] tripeptide following the stop codon. However, C. geo1 (SEQ ID 003) has incorporated the tripeptide into the mature sequence, thereby increasing the C-terminal diversity of this peptide family.

### Example 8

### Discovery of C. Geo3

A mass of 3094.35 Da was identified in an active SubFr 33.6 of conus geographus (40% block of hNav1.7). A sequence of a peptide was identified (SEQ ID 025) in the transcriptome data for Conus geographus, characterized by the same mass. It was then synthesized and folded in the presence of reduced and oxidized gluthatione. Three peaks of the same, desired mass were collected and tested against hNav1.7 and rNav1.7. In both cases peptide was not active.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

1. An isolated peptide having a sequence GWCGDOGATC GKLRLYCCSG FCX₂₃C₂₄X₂₅TKTC-X₃₀̂ (SEQ ID 001), where O is hydroxyproline, X₂₃ is aspartic acid, asparagine, or carboxyglutamic acid, C₂₄ is cysteine or a substituted cysteine, X₂₅ is tyrosine or aspartic acid, X₃₀ is a peptide from 0 to 6 amino acids, and ̂ is a carboxylated C-terminus.

2. The isolated peptide of claim 1, wherein X is KDKSSA (SEQ ID 002).

3. The isolated peptide of claim 1, wherein the peptide is a synthetic peptide.

4. The isolated peptide of claim 1, further comprising a label.

5. The isolated peptide of claim 1, wherein the label is a fluorescent label.

6. The isolated peptide of claim 1, which is modified to contain an O-glycan, an S-glycan or an N-glycan.

7. The isolated peptide of claim 1, wherein C₂₄ is a free-thiol substituted cysteine.

8. The isolated peptide of claim 1, wherein C₂₄ forms a dimer with a second peptide of SEQ ID 001.

9. The isolated peptide of claim 1, wherein C₂₄ is replaced by an alternative amino acid residue.

10. The isolated peptide of claim 1, wherein C₂₄ is reversibly modified with a molecule through a disulfide linkage.

11. The isolated peptide of claim 10, wherein the molecule includes a member selected from the group consisting of glutathione, cysteine, cysteamine, DTNB, selenocysteine, selenoglutathione, and any product of a reaction of C₂₄ with an alkanethiosulfonate reagent or a thiosulfate reagent, and combinations thereof.

12. The isolated peptide of claim 1, wherein C₂₄ is irreversibly modified with a molecule.

13. The isolated peptide of claim 12, wherein the molecule includes a member selected from the group consisting of acetamidomethyl, products of a reaction of C₂₄ with maleimides, vinyl sulfones and related α,β-unsaturated systems, β-haloethylamine, α-halocarbonyls, or a combination thereof.

14. The isolated peptide of claim 1, where X₂₃ is aspartic acid, C₂₄ is an un-substituted cysteine, and X₂₅ is tyrosine.

15. The isolated peptide of claim 14, wherein X₃₀ is SEQ ID 002.

16. The isolated peptide of claim 1, wherein X₂₃ is aspartic acid, C₂₄ is substituted with cystamine, and X₂₅ is tyrosine.

17. The isolated peptide of claim 16, wherein X₃₀ is SEQ ID 002.

18. An isolated peptide having 7 cysteine residues and a sequence of X₁X₂C X₄X₅X₆X₇X₈X₉C X₁₁X₁₂X₁₃X₁₄X₁₅X₁₆CCX₁₉X₂₀X₂₁C X₂₃C₂₄X₂₅X₂₆X₂₇X₂₈Ĉ (SEQ ID 033), wherein X₁₋₂, X₄₋₉, X₁₁₋₁₆, X₁₉₋₂₁, X₂₃, and X₂₅₋₂₈ are each independently any amino acid, C₂₄ is cysteine or a substituted cysteine, and ̂ is a carboxylated C-terminus.

19. The isolated peptide of claim 18, wherein the peptide further includes a fluorescent label.

20. The isolated peptide of claim 18, wherein the peptide is a synthetic peptide.

21-44. (canceled)


SEQUENCE LISTING (33 sequences)

SEQ ID NO: 1 - protein, 30 aa, Conus geographus
  MOD_RES (6)..(6): 3Hyp
GWCGDAGATCGKLRLYCCSGFCXCXTKTCX

SEQ ID NO: 2 - protein, 6 aa, Conus geographus
  NON_TER (1)..(1)
KDKSSA

SEQ ID NO: 3 - protein, 35 aa, Conus geographus
  MOD_RES (6)..(6): 3Hyp
GWCGDAGATCGKLRLYCCSGFCDCYTKTCKDKSSA

SEQ ID NO: 4 - protein, 35 aa, Conus geographus; identical to SEQ ID NO: 3
  MOD_RES (6)..(6): 3Hyp

SEQ ID NO: 5 - protein, 35 aa, Conus geographus
  MOD_RES (6)..(6): 3Hyp
GWCGDAGATCGKLRLYCCSGFCDSYTKTCKDKSSA

SEQ ID NO: 6 - protein, 35 aa, Conus geographus
  MOD_RES (6)..(6): 3Hyp
GWCGDAGATCGKLRLYCCSGFCDKYTKTCKDKSSA

SEQ ID NO: 7 - protein, 35 aa, Conus geographus
  MOD_RES (6)..(6): 3Hyp
GWCGDAGATCGKLRLYCCSGFCDEYTKTCKDKSSA

SEQ ID NO: 8 - protein, 35 aa, Conus geographus
  MOD_RES (6)..(6): 3Hyp
GWCGDAGATCGKLRLYCCSGFCDCYTGTCKDKSSA

SEQ ID NO: 9 - protein, 35 aa, Conus geographus; identical to SEQ ID NO: 3
  DISULFID (24)..(24): Disulfide link with Glutathione

SEQ ID NO: 10 - protein, 35 aa, Conus geographus; identical to SEQ ID NO: 3
  MOD_RES (6)..(6): 3Hyp

SEQ ID NO: 11 - protein, 35 aa, Conus geographus; identical to SEQ ID NO: 3
  MOD_RES (6)..(6): 3Hyp

SEQ ID NO: 12 - protein, 35 aa, Conus geographus; identical to SEQ ID NO: 3
  MOD_RES (6)..(6): 3Hyp

SEQ ID NO: 13 - protein, 35 aa, Conus geographus; identical to SEQ ID NO: 3
  MOD_RES (6)..(6): 3Hyp

SEQ ID NO: 14 - protein, 35 aa, Conus geographus; identical to SEQ ID NO: 3
  MOD_RES (6)..(6): 3Hyp

SEQ ID NO: 15 - protein, 35 aa, Conus geographus; identical to SEQ ID NO: 3
  MOD_RES (6)..(6): 3Hyp

SEQ ID NO: 16 - protein, 35 aa, Conus geographus; identical to SEQ ID NO: 3
  MOD_RES (6)..(6): 3Hyp

SEQ ID NO: 17 - protein, 35 aa, Conus geographus; identical to SEQ ID NO: 3
  MOD_RES (6)..(6): 3Hyp

SEQ ID NO: 18 - protein, 35 aa, Conus geographus; identical to SEQ ID NO: 3
  MOD_RES (6)..(6): 3Hyp

SEQ ID NO: 19 - protein, 35 aa, Conus geographus; identical to SEQ ID NO: 3
  MOD_RES (6)..(6): 3Hyp

SEQ ID NO: 20 - protein, 35 aa, Conus geographus
  MOD_RES (6)..(6): 3Hyp
GWCGDAGATCGKLRLYCCSGFCNCYTKTCKDKSSA

SEQ ID NO: 21 - protein, 35 aa, Conus geographus
  MOD_RES (6)..(6): 3Hyp
GWCGDAGATCGKLRLYCCSGFCDCDTKTCKDKSSA

SEQ ID NO: 22 - protein, 30 aa, Conus geographus
  MOD_RES (6)..(6): 3Hyp
GWCGDAGATCGKLRLYCCSGFCDCYTKTCX

SEQ ID NO: 23 - protein, 35 aa, Conus geographus; identical to SEQ ID NO: 3
  MOD_RES (6)..(6): 3Hyp

SEQ ID NO: 24 - protein, 31 aa, Conus geographus
DWCGDAGDACGTLKLRCCSGLCNQYSGTCTG

SEQ ID NO: 25 - protein, 28 aa, Conus geographus
CVGRDSKCGPPPCCMGMTCNYERVRKCT

SEQ ID NO: 26 - protein, 29 aa, Conus geographus
  misc_feature (2)..(2): Xaa can be any naturally occurring amino acid
GXCCGDAGATCKLRLYCCSGFCDCYTCTC

SEQ ID NO: 27 - protein, 35 aa, Conus geographus
GWCGDPGATCGKLRLYCCSGFCDCYTKTCKDKSSA

SEQ ID NO: 28 - protein, 34 aa, Conus geographus
  MOD_RES (6)..(6): 3Hyp
GWCGDAGATCGKLRLYCCSGFCDCYTKTCKDKSA

SEQ ID NO: 29 - protein, 34 aa, Conus geographus; identical to SEQ ID NO: 28
  MOD_RES (6)..(6): 3Hyp

SEQ ID NO: 30 - protein, 69 aa, Conus geographus
GGTQHRALRSTIKLSLLRQHRGWCGDPGATCGKLRLYCCSGFCDCYTKTCKDKSSASSPSVLYPFLPES

SEQ ID NO: 31 - protein, 8 aa, Conus geographus
  NON_TER (1)..(1)
  MUTAGEN (2)..(2): alkylated with 4-vinylpyridine
DCYTKTCK

SEQ ID NO: 32 - protein, 30 aa, Conus geographus
  UNSURE (1)..(2)
  NON_TER (30)..(30)
XXCGDAGDACGTLKLRCCSGLCNQYSGTCS

SEQ ID NO: 33 - protein, 29 aa, Conus geographus
  VARIANT (1)..(2): Can each independently be any amino acid that allows functionality of the resulting peptide
XXCXXXXXXCXXXXXXCCXXXCXCXXXXC